(19) United States
(12) Patent Application Publication
(10) Pub. No.: US 2021/0085926 A1
(43) Pub. Date: Mar. 25, 2021

(54) CATHETER STABILIZATION DEVICE

(71) Applicant: Eong Sok KIM, Wonju-si (KR)

(72) Inventor: Eong Sok KIM, Wonju-si (KR)

(21) Appl. No.: 16/707,353

(22) Filed: Dec. 9, 2019

Related U.S. Application Data
(63) Continuation of application No. PCT/KR2019/017324, filed on Dec. 9, 2019.

(30) Foreign Application Priority Data
Sep. 23, 2019 (KR) 10-2019-0117046
Sep. 23, 2019 (KR) 10-2019-0117054
Dec. 3, 2019 (KR) 10-2019-0159080
Dec. 9, 2019 (KR) 10-2019-0162473

(51) Int. Cl. A61M 25/02

(52) U.S. Cl. CPC A61M 25/02; A61M 2025/0246; A61M 2025/024

(57) ABSTRACT

Provided is a two-way clamping catheter stabilization device and a hook-type catheter stabilization device, and the two-way clamping catheter stabilization device includes a base part having a top surface on which a fixing target catheter is mounted, a pin part including a first pin and a second pin protruding upwards from the top surface of the base part, a first coupling part placed on a right side of the base part and coupled to the base part to be slidably moved in two ways along left and right directions of the base part, and a second coupling part placed on a left side of the base part and coupled to the base part to be slidably moved in two ways along the left and right directions of the base part, and when the first coupling part and the second coupling part slide to be closer to each other, they are placed in a locked state in which an upward movement of the mounted fixing target catheter is limited.

### TECHNICAL FIELD

The present disclosure relates to a catheter stabilization device and particularly, to a two-way clamping catheter stabilization device and a hook-type catheter stabilization device.

### BACKGROUND

A catheter is a device that can be inserted in the body of a subject to inject a medicine or induce the discharge of fluid present in the body and can be manufactured in various forms depending on the site and purpose of treatment.

The catheter moves along the movements of the subject, which may cause inconvenience to the subject or may cause inflammation or separation from the insertion site in severe cases, and, thus, the catheter located outside the body needs to be fixed. Conventionally, the catheter has been fixed to the body by stitching a part of the catheter located outside the body to the skin or by using skin adhesive means such as a Band-Aid. However, in many cases, the stitched skin dies and the fixed part becomes loose, and the stitched site may be scarred. Also, the stitched site may be torn out by a strong external force.

The Band-Aid has weak fixing power and thus is highly likely to make the catheter come out, and during a replacement operation, an adhesive adhering to the catheter may cause the tube to be pulled out.

Further, in a conventional catheter stabilization device, a method of placing a catheter and covering the catheter from above with a fixing cover for suppressing the separation of the catheter to fix the catheter has been used. However, according to the method of covering the catheter from above, after placing the catheter, an operator needs to apply force for pressing the fixing cover downwards or force for fixing the fixing cover on the catheter to the body of the subject in order to fix the catheter. Therefore, unnecessarily great force is applied to the subject such as a patient and may cause unexpected damage to the body of the subject. Also, when the conventional catheter stabilization device is used, if the catheter stabilization device is held in a hand in order not to apply force to the body of the subject, the catheter becomes more likely to be separated.

The background technology of the present disclosure is disclosed in Korean Patent No. 10-1622245.

### DISCLOSURE OF THE INVENTION

### Problems to be Solved by the Invention

In view of the foregoing, the present disclosure provides a two-way clamping catheter stabilization device that provides fixing and locking to a catheter in a sliding manner and enables an operator to fix the catheter just by applying force in left and right directions and thus can reduce external force applied to the body of a subject when the catheter is fixed to a part of the body of the subject. Also, the two-way clamping catheter stabilization device is not fixed by being held in a hand but can be fixed stably at a fixed position and thus can remove the risk of separation during installation.

In view of the foregoing, the present disclosure provides a hook-type catheter stabilization device that provides fixing and locking to a catheter or a tube by hooking and clamping a hook made of a bendable and elastically deformable material and enables an operator to fix the catheter by applying force for moving the hook in a longitudinal direction and thus can reduce external force applied to the body of a subject when the catheter is fixed to a part of the body of the subject. Also, the hook-type catheter stabilization device is not fixed by being held in a hand but can be fixed stably at a fixed position and thus can remove the risk of separation during installation.

In view of the foregoing, the present disclosure provides a two-way clamping catheter stabilization device and a hook-type catheter stabilization device that have excellent fixing power and do not apply force toward the body of a patient during installation. Also, the two-way clamping catheter stabilization device and the hook-type catheter stabilization device can remove the risk of separation of a catheter and fix the catheter suitably for the body of the subject.

However, problems to be solved by the present disclosure are not limited to the above-described problems. There may be other problems to be solved by the present disclosure.

### Means for Solving the Problems

As a technical means for solving the above-described technical problems, a two-way clamping catheter stabilization device according to an exemplary embodiment of the present disclosure includes a base part having a top surface on which a fixing target catheter is mounted, a pin part including a first pin and a second pin protruding upwards from the top surface of the base part, a first coupling part placed on a right side of the base part and coupled to the base part to be slidably moved in two ways along left and right directions of the base part, and a second coupling part placed on a left side of the base part and coupled to the base part to be slidably moved in two ways along the left and right directions of the base part.

Further, when the first coupling part and the second coupling part slide to be closer to each other, they may be placed in a locked state in which an upward movement of the mounted fixing target catheter is limited.

Further, the first pin and the second pin may be inserted into holes formed in wing-type members of the fixing target catheter.

Also, in the locked state, the upward movement of the fixing target catheter may be limited by letting the wing-type members to move between a bottom side of the first coupling part and the second coupling part and a top side of the base part.

Further, a first groove that is dented in a right direction to cover a top portion of the first pin in the locked state may be formed in the first coupling part, and a second groove that is dented in a left direction to cover a top portion of the second pin in the locked state may be formed in the second coupling part.

Furthermore, the fixing target catheter may be separated when the wing-type members are separated upwardly from the first pin and the second pin in an unlocked state in which the first coupling part and the second coupling part slide to be farther from each other.—claim 4

Moreover, a first hole in which the first pin is to be inserted and a second hole in which the second pin is to be inserted may be formed in the base part, and the first pin may partially protrude upwards from a bottom side of the base part through the first hole and the second pin may partially protrude upwards from a bottom side of the base part through the second hole.

Also, the pin part may include a connection member that connects the first pin and the second pin, and a dented coupling groove corresponding in shape to the connection member may be formed in a bottom surface of the base part.

Further, the connection member may be provided including a curved portion in at least a partial region.

Furthermore, the base part may include a protrusion member configured to suppress a downward separation of the connection member inserted in the coupling groove from the base part.

Moreover, the protrusion member may protrude toward an inside of the coupling groove and may have a top portion that protrudes more than a bottom portion.

Also, the base part may include a first support member protruding in a right direction on a lower right side of the base part and a second support member protruding in a left direction on a lower left side of the base part.

Further, the first support member may include a first hook member that protrudes upwards, and a first hook groove that is to be hooked and clamped to the first hook member in the locked state may be formed in the first coupling part.

Furthermore, the second support member may include a second hook member that protrudes upwards, and a second hook groove that is to be hooked and clamped to the second hook member in the locked state may be formed in the second coupling part.

Moreover, the base part may include a first male rail extended in the left and right directions on a front end of the base part and a second male rail extended in the left and right directions on a rear end of the base part.

Also, the first coupling part may include a first female rail corresponding to the first male rail and the second male rail, and the second coupling part may include a second female rail corresponding to the first male rail and the second male rail.

Further, the first coupling part may be coupled to the base part in order for the first female rail to be slidably moved in the left and right directions along the first male rail and the second male rail, and the second coupling part may be coupled to the base part in order for the second female rail to be slidably moved in the left and right directions along the first male rail and the second male rail.

Furthermore, the first coupling part may include a first limit block protruding toward the left direction on a top portion of the first coupling part and the second coupling part may include a second limit block protruding toward the right direction on a top portion of the second coupling part.

Moreover, the first limit block and the second limit block may be provided to suppress an upward separation of the fixing target catheter in the locked state.

Also, the first limit block and the second limit block may protrude from a region leaning from forwards or backwards center in forward and backward directions of the top portion of the first coupling part and the top portion of the second coupling part, respectively.

Meanwhile, a hook-type catheter stabilization device according to an exemplary embodiment of the present disclosure includes a base part having a pair of hook members that protrude from one side in a longitudinal direction and are located at a distance from each other in a widthwise direction, a pin part including a first pin and a second pin protruding upwards from a top surface of the base part, and a hook unit having the other end to be coupled to the other side of the base part in the longitudinal direction and one end including a pair of hook grooves to which the pair of hook members are to be hooked and clamped, respectively.

Further, the hook unit may be made of a material that is bendable and elastically deformable in the longitudinal direction.

Furthermore, a length of the hook unit may be set in order for the pair of hook grooves to be located closer to the other side in the longitudinal direction than the pair of hook members when the one end is moved to the one side in the longitudinal direction in a state in which the hook unit is not elastically deformed in the longitudinal direction.

Moreover, when the one end of the hook unit is moved to the one side in the longitudinal direction in order for the hook unit to be elastically deformed at least in part in the longitudinal direction and for the pair of hook grooves to be hooked and clamped to the pair of hook members, a fixing target catheter mounted on the top surface of the base part may be placed in a locked state in which an upward movement of the fixing target catheter is limited.

Also, the pair of hook grooves may be dented toward the center of the hook unit in the widthwise direction in order to correspond to the distance between the pair of hook members.

Further, in the locked state, a region leaning toward the one side of the hook unit from the pair of hook grooves of the hook unit may be supported by the pair of hook members to maintain the locked state.

Furthermore, the first pin and the second pin may be inserted into holes formed in wing-type members of the fixing target catheter, and in the locked state, the upward movement of the fixing target catheter may be limited by letting the wing-type members to move between a bottom side of the hook unit and a top side of the base part.

Moreover, the fixing target catheter may be separated when the wing-type members are separated upwardly from the first pin and the second pin in an unlocked state in which hooking and clamping of the pair of hook grooves and the pair of hook members is canceled.—claim 19

Further, a first hole in which the first pin is to be inserted and a second hole in which the second pin is to be inserted may be formed in the base part, and the first pin may partially protrude upwards from a bottom side of the base part through the first hole and the second pin may partially protrude upwards from a bottom side of the base part through the second hole.

Furthermore, the pin part may include a connection member that connects the first pin and the second pin.

Also, a dented coupling groove corresponding in shape to the connection member may be formed in a bottom surface of the base part.

Further, the connection member may be provided including a curved portion in at least a partial region.

Furthermore, the base part may include a protrusion member configured to suppress a downward separation of the connection member inserted in the coupling groove from the base part.

Moreover, the protrusion member may protrude toward an inside of the coupling groove and may have a top portion that protrudes more than a bottom portion.

The above-described exemplary embodiments are provided by way of illustration only and should not be construed as liming the present disclosure. Besides the above-described exemplary embodiments, there may be additional exemplary embodiments described in the accompanying drawings and the detailed description.

### Effects of the Invention

According to the above-described means for solving the problems, it is possible to provide a two-way clamping catheter stabilization device that provides fixing and locking to a catheter in a sliding manner and enables an operator to fix the catheter just by applying force in left and right directions and thus can reduce external force applied to the body of a subject when the catheter is fixed to a part of the body of the subject.

According to the above-described means for solving the problems, it is possible to provide a hook-type catheter stabilization device that provides fixing and locking to a catheter by hooking and clamping a hook made of a bendable and elastically deformable material and enables an operator to fix the catheter by applying force for moving the hook in a longitudinal direction and thus can reduce external force applied to the body of a subject when the catheter is fixed to a part of the body of the subject.

According to the above-described means for solving the problems, the operator can fix a fixing target catheter just by closing a first coupling part and a second coupling part of the two-way clamping catheter stabilization device together toward the inside or pulling a hook unit of the hook-type catheter stabilization device, and, thus, convenience can be improved.

### BRIEF DESCRIPTION OF THE DRAWINGS

FIG. 1 is a schematic perspective view of a two-way clamping catheter stabilization device according to an exemplary embodiment of the present disclosure;

FIG. 2 is a plan view of the two-way clamping catheter stabilization device when viewed from above according to an exemplary embodiment of the present disclosure;

FIG. 3 is a diagram illustrating an example of the shape of a fixing target catheter mounted on a catheter stabilization device according to an exemplary embodiment of the present disclosure;

FIG. 4 is a left and right cross-sectional view of the two-way clamping catheter stabilization device according to an exemplary embodiment of the present disclosure;

FIG. 5 is a front view of the two-way clamping catheter stabilization device according to an exemplary embodiment of the present disclosure;

FIG. 6 is a schematic perspective view of a base part of the two-way clamping catheter stabilization device according to an exemplary embodiment of the present disclosure;

FIG. 7 is a bottom view of the base part of the two-way clamping catheter stabilization device when viewed from below according to an exemplary embodiment of the present disclosure;

FIG. 8 is a schematic perspective view of a first coupling part of the two-way clamping catheter stabilization device according to an exemplary embodiment of the present disclosure;

FIG. 9 is a schematic perspective view of a second coupling part of the two-way clamping catheter stabilization device according to an exemplary embodiment of the present disclosure;

FIG. 10 is a perspective view schematically illustrating a state before a fixing target catheter is inserted into a two-way clamping catheter stabilization device according to another exemplary embodiment of the present disclosure;

FIG. 11 is a perspective view illustrating a state in which a first coupling part and a second coupling part are moved and fixed to fix the fixing target catheter inserted into the two-way clamping catheter stabilization device according to another exemplary embodiment of the present disclosure;

FIG. 12 is a side view of the two-way clamping catheter stabilization device when viewed from lateral side according to another exemplary embodiment of the present disclosure;

FIG. 13A is a cross-sectional view provided to explain an anti-retraction/opening structure in a state in which the fixing target catheter is not fixed by the two-way clamping catheter stabilization device according to another exemplary embodiment of the present disclosure;

FIG. 13B is a cross-sectional view provided to explain an anti-retraction/opening structure in a state in which the fixing target catheter is fixed by the two-way clamping catheter stabilization device according to another exemplary embodiment of the present disclosure;

FIG. 14 is a schematic perspective view of a hook-type catheter stabilization device according to an exemplary embodiment of the present disclosure;

FIG. 15 is a bottom view of the hook-type catheter stabilization device when viewed from below according to an exemplary embodiment of the present disclosure;

FIG. 16 is a side view of the hook-type catheter stabilization device when viewed from lateral side according to an exemplary embodiment of the present disclosure;

FIG. 17 is a schematic perspective view of a base part of the hook-type catheter stabilization device according to an exemplary embodiment of the present disclosure;

FIG. 18 is a bottom view of the base part of the hook-type catheter stabilization device when viewed from below according to an exemplary embodiment of the present disclosure;

FIG. 19 is a perspective view schematically illustrating a hook-type catheter stabilization device according to another exemplary embodiment of the present disclosure; and

FIG. 20 is a plan view of the hook-type catheter stabilization device when viewed from a predetermined direction according to another exemplary embodiment of the present disclosure.

### BEST MODE FOR CARRYING OUT THE INVENTION

Hereinafter, embodiments of the present disclosure will be described in detail with reference to the accompanying drawings so that the present disclosure may be readily implemented by those skilled in the art. However, it is to be noted that the present disclosure is not limited to the embodiments but can be embodied in various other ways. In drawings, parts irrelevant to the description are omitted for the simplicity of explanation, and like reference numerals denote like parts through the whole document.

Through the whole document, the term “connected to” or “coupled to” that is used to designate a connection or coupling of one element to another element includes both a case that an element is “directly connected or coupled to” another element and a case that an element is “electronically connected or coupled to” or “indirectly connected or coupled to” another element via still another element.

Through the whole document, the terms “on”, “above”, “on an upper end”, “below”, “under”, and “on a lower end” that are used to designate a position of one element with respect to another element include both a case that the one element is adjacent to the other element and a case that any other element exists between these two elements.

Further, through the whole document, the term “comprises or includes” and/or “comprising or including” used in the document means that one or more other components, steps, operation and/or existence or addition of elements are not excluded in addition to the described components, steps, operation and/or elements unless context dictates otherwise.

Hereinafter, a two-way clamping catheter stabilization device of the present disclosure will be described in detail with reference to FIG. 1 to FIG. 13.

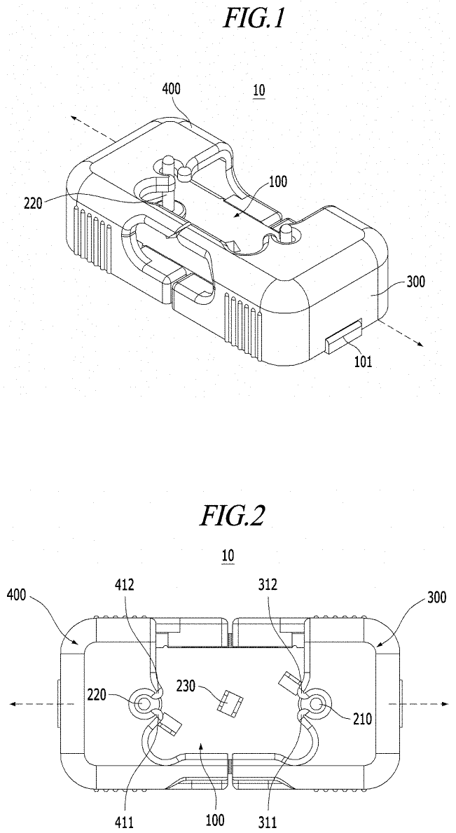

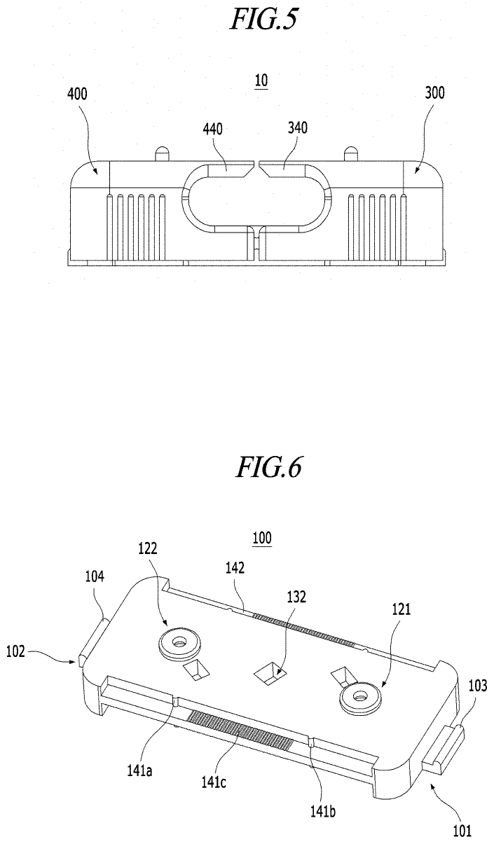

For reference, any terms referring to direction or location (top surface, top side, upward, bottom side, bottom portion, left and right directions, forward and backward directions, forwards, backwards, etc.) relating to a two-way clamping catheter stabilization device 10 according to an exemplary embodiment of the present disclosure to be described below with reference to FIG. 1 to FIG. 9 are based on the placement of components shown in the drawings. For example, in FIG. 1 and FIG. 6, forward and backward directions may be from 2 o'clock to 8 o'clock direction, left and right directions may be from 4 o'clock to 10 o'clock direction, and upward and downward directions may be from 12 o'clock to 6 o'clock direction. Also, in FIG. 2, forward and backward directions may be from 12 o'clock to 6 o'clock direction, left and right directions may be from 3 o'clock to 9 o'clock direction, and upward and downward directions may be directions of normal to the drawing sheet.

However, this direction setting may be changed depending on the placement of the device of the present disclosure. For example, the device of the present disclosure may be placed such that an upward direction in FIG. 1 faces left and right directions if necessary. For another example, the device of the present disclosure may be placed such that an upward direction in FIG. 1 faces a slanted direction.

FIG. 1 is a schematic perspective view of a two-way clamping catheter stabilization device according to an exemplary embodiment of the present disclosure, and FIG. 2 is a plan view of the two-way clamping catheter stabilization device when viewed from above according to an exemplary embodiment of the present disclosure.

Referring to FIG. 1 and FIG. 2, the two-way clamping catheter stabilization device 10 according to an exemplary embodiment of the present disclosure (hereinafter, referred to as “catheter stabilization device 10”) may include a base part 100, a pin part 200, a first coupling part 300 and a second coupling part 400.

A fixing target catheter 20 may be mounted on a top surface of the base part 100. According to an exemplary embodiment of the present disclosure, the fixing target catheter 20 may be mounted on the center of the base part 100 in the left and right directions so as to be extended along the forward and backward directions of the base part 100. In other words, the fixing target catheter 20 may be mounted (placed) on the catheter stabilization device 10 so that a longitudinal direction of the fixing target catheter 20 agrees with the forward and backward directions of the catheter stabilization device 10.

According to an exemplary embodiment of the present disclosure, although not illustrated in the drawings, an adhesion means for adhesion of the present catheter stabilization device 10 to the body of a subject (for example, the skin of the subject) may be provided in at least a part of the base part 100, but the present disclosure is not limited thereto.

The pin part 200 may include a first pin 210 and a second pin 220 protruding upwards from the top surface of the base part 100.

The first coupling part 300 may be placed on the right side of the base part 100 and coupled to the base part 100 to be slidably moved in two ways along the left and right directions of the base part 100.

The second coupling part 400 may be placed on the left side of the base part 100 and coupled to the base part 100 to be slidably moved in two ways along the left and right directions of the base part 100.

Further, when the first coupling part 300 and the second coupling part 400 slide to be closer to each other, they may be placed in a locked state in which an upward movement of the mounted fixing target catheter 20 is limited.

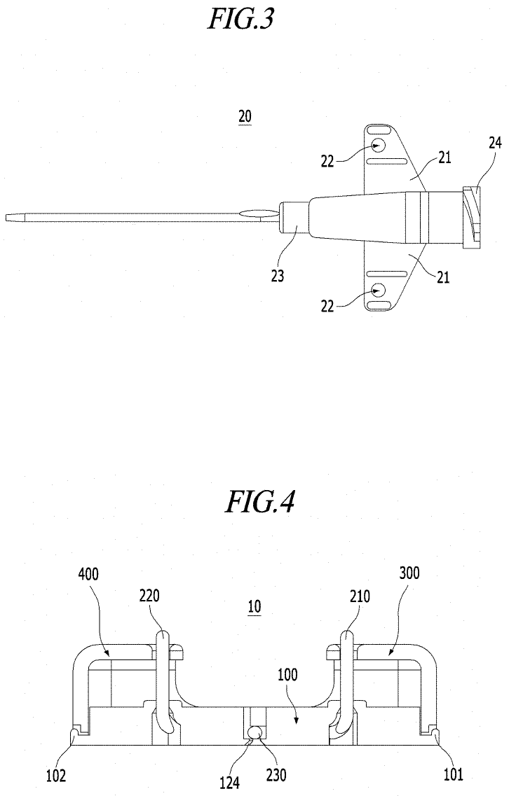

FIG. 3 is a diagram illustrating an example of the shape of a fixing target catheter mounted on a catheter stabilization device according to an exemplary embodiment of the present disclosure.

Referring to FIG. 3, the fixing target catheter 20 to be mounted on the catheter stabilization device 10 according to an exemplary embodiment of the present disclosure may include wing-type members 21 provided on both sides, respectively, of the fixing target catheter 20 in a widthwise direction (for example, from 12 o'clock to 6 o'clock direction in FIG. 3). Further, holes 22 may be formed in the respective wing-type members 21. Furthermore, referring to FIG. 3, the fixing target catheter 20 may include a front part 23 to be connected to a needle (not illustrated) for infiltration and a connection part 24 to be connected to a tube member (not illustrated) that is a passage for injecting a chemical solution into the body.

According to an exemplary embodiment of the present disclosure, the first pin 210 and the second pin 220 may be inserted into the holes 22 formed in the wing-type members 21, respectively, of the fixing target catheter 20. Further, according to an exemplary embodiment of the present disclosure, in the locked state (in other words, in a state in which the first coupling part 300 and the second coupling part 400 slide to be closer to each other), the upward movement of the fixing target catheter 20 may be limited by letting the wing-type members 21 to move between a bottom side of the first coupling part 300 and the second coupling part 400 and a top side of the base part 100 along the first pin 210 or the second pin 220.

Specifically, a first groove 310 that is dented in a right direction to cover a top portion of the first pin 210 in the locked state may be formed in the first coupling part 300. Also, a second groove 410 that is dented in a left direction to cover a top portion of the second pin 220 in the locked state may be formed in the second coupling part 400.

According to an exemplary embodiment of the present disclosure, the fixing target catheter 20 may be separated from the catheter stabilization device 10 when the wing-type members 21 of the fixing target catheter 20 are separated upwardly from the first pin 210 and the second pin 220 in an unlocked state in which the first coupling part 300 and the second coupling part 400 slide to be farther from each other.

Also, according to an exemplary embodiment of the present disclosure, a first hole 121 in which the first pin 210 is to be inserted and a second hole 122 in which the second pin 220 is to be inserted may be formed in the base part 100. Herein, the first pin 210 may partially protrude upwards from a bottom side of the base part 100 through the first hole 121. Likewise, the second pin 220 may partially protrude upwards from a bottom side of the base part 100 through the second hole 122.

Further, according to an exemplary embodiment of the present disclosure, the pin part 200 may include a connection member 230 that connects the first pin 210 and the second pin 220. Furthermore, a dented coupling groove 123 corresponding in shape to the connection member 230 may be formed in a bottom surface of the base part 100.

According to an exemplary embodiment of the present disclosure, the connection member 230 of the pin part 200 may be provided including a curved portion in at least a partial region. For example, the connection member 230 may be provided in an S-shape between the first pin 210 and the second pin 220. Uke the connection member 230, the coupling groove 123 may be provided including a curved portion in at least a partial region so as to correspond in shape to the connection member 230.

According to an exemplary embodiment of the present disclosure, the connection member 230 and the coupling groove 123 corresponding to the connection member 230 have a shape including a curved portion in at least a partial region, and, thus, even if unexpected external force is applied to a partial region of the pin part 200 inserted into the base part 100, the external force works along an in-plane direction of the connection member 230. Therefore, it is possible to suppress leaning of the pin part 200 to any one side or a difference in the degree of protrusion between the first pin 210 and the second pin 220. Eventually, it is possible to suppress the separation of the pin part 200 from the base part 100.

Further, according to an exemplary embodiment of the present disclosure, the base part 100 may include at least one protrusion member 124 configured to suppress a downward separation of the connection member 230 inserted in the coupling groove 123 from the base part 100. Specifically, the protrusion member 124 may protrude toward an inside of the coupling groove 123 and may have a top portion that protrudes more than a bottom portion. In other words, the protrusion member 124 may include a slanted surface in at least a partial region along the upward and downward directions. The slanted surface may be provided in order for the connection member 230 to be easily coupled to (mounted on) the coupling groove 123 while moving along the slanted surface from the bottom side to the top side of the base part 100.

Furthermore, if the connection member 230 passes through a region where the protrusion member 124 protrudes and is inserted to be located on a top side of the protrusion member 124, the connection member 230 is supported upwards by the protrusion member 124. Therefore, it is possible to suppress a downward separation of the pin part 200 from the base part 100.

Moreover, according to an exemplary embodiment of the present disclosure, desirably, the pin part 200 may be made of metal at least in part in order for the first pin 210 and the second pin 220 not to be deformed even when the fixing target catheter 20 placed on the catheter stabilization device 10 makes an unexpected movement.

Also, according to an exemplary embodiment of the present disclosure, the base part 100 may include a first support member 101 protruding in the right direction on a lower right side of the base part 100. Further, the base part 100 may include a second support member 102 protruding in the left direction on a lower left side of the base part 100. Further, the first support member 101 may include a first hook member 103 that protrudes upwards. Furthermore, the second support member 102 may include a second hook member 104 that protrudes upwards. A first hook groove (not illustrated) that corresponds in shape to the first hook member 103 so as to be hooked and clamped to the first hook member 103 in the locked state may be formed in the first coupling part 300. According to an exemplary embodiment of the present disclosure, the first hook groove (not illustrated) may be dented upwards on a lower right side of the first coupling part 300 so as to correspond in shape to the upwardly protruding first hook member 103. Likewise, a second hook groove (not illustrated) that corresponds in shape to the second hook member 104 so as to be hooked and clamped to the second hook member 104 in the locked state may be formed in the second coupling part 400. According to an exemplary embodiment of the present disclosure, the second hook groove (not illustrated) may be dented upwards on a lower left side of the second coupling part 400 so as to correspond in shape to the upwardly protruding second hook member 104.

Since the first hook member 103 of the base part 100 is hooked and clamped to the first hook groove of the first coupling part 300 in the locked state, the first coupling part 300 can be fixed to the base part 100 as long as extra external force for moving the first coupling part 300 to be farther from the second coupling part 400 is not applied. Likewise, since the second hook member 104 of the base part 100 is hooked and clamped to the second hook groove of the second coupling part 400 in the locked state, the second coupling part 400 can be fixed to the base part 100 if extra external force for moving the second coupling part 400 to be farther from the first coupling part 300 is not applied.

FIG. 4 is a left and right cross-sectional view of the two-way clamping catheter stabilization device according to an exemplary embodiment of the present disclosure, and FIG. 5 is a front view of the two-way clamping catheter stabilization device according to an exemplary embodiment of the present disclosure.

Referring to FIG. 4 and FIG. 5, the first coupling part 300 may include a first limit block 340 protruding toward the left direction on a top portion of the first coupling part. Also, the second coupling part 400 may include a second limit block 440 protruding toward the right direction on a top portion of the second coupling part 400. According to an exemplary embodiment of the present disclosure, the first limit block 340 and the second limit block 440 serve (function) to hold the fixing target catheter 20 placed at a predetermined insertion site in the body not to move even when the connection part 23 is moved unexpectedly by pressing the front part 23 of the fixing target catheter 20.

Further, referring to FIG. 2 and FIG. 5, the first limit block 340 and the second limit block 440 according to an exemplary embodiment of the present disclosure may protrude from a region leaning from forwards or backwards center in forward and backward directions of the top portion of the first coupling part 300 and the top portion of the second coupling part 400, respectively. For example, referring to FIG. 2, the first limit block 340 and the second limit block 440 may protrude along the forward and backward directions from front ends in the forward and backward directions of the top portion of the first coupling part 300 and the top portion of the second coupling part 400, respectively, but may not be limited thereto.

According to an exemplary embodiment of the present disclosure, since the first limit block 340 and the second limit block 440 protrude from a region leaning from forwards or backwards center in the forward and backward directions of the top portion of the first coupling part 300 and the top portion of the second coupling part 400, respectively, a region of the catheter stabilization device 10 where the fixing target catheter 20 is placed in the locked state can be exposed to the outside, and, thus, the operator can easily check the status of the placed fixing target catheter 20 with the naked eye. If the operator separates the placed fixing target catheter 20 from the catheter stabilization device 10 (in other words, to put it in the unlocked state), the fixing target catheter 20 can be easily separated due to a large exposed region above the region where the fixing target catheter 20 is placed.

Also, referring to G. 5, since the first limit block 340 and the second limit block 440 protrude from the top portion of the first coupling part 300 and the top portion of the second coupling part 400, respectively, in the locked state, an empty space where the fixing target catheter 20 can pass through may be formed under the first limit block 340 and the second limit block 440.

Further, according to an exemplary embodiment of the present disclosure, the first limit block 340 and the second limit block 440 may be made of a flexible material which can be bent to a predetermined extent in order to press the fixing target catheter 20 downwards to suppress an upward separation of the fixing target catheter 20 even when the fixing target catheter 20 mounted on the catheter stabilization device 10 is changed in diameter. For example, even if the fixing target catheter 20 having a larger dimeter than the vertical height of the empty space formed under the first limit block 340 and the second limit block 440 is placed on the catheter stabilization device 10, a part of the first limit block 340 and a part of the second limit block 440 (e.g., a protruding end of the first limit block 340 and a protruding end of the second limit block 440) can accommodate the fixing target catheter 20 having a large diameter while partially moving upwards. In other words, the height of the space under the first limit block 340 and the second limit block 440 can increase depending on the diameter of the fixing target catheter 20 in order to accommodate the fixing target catheters 20 of various diameters.

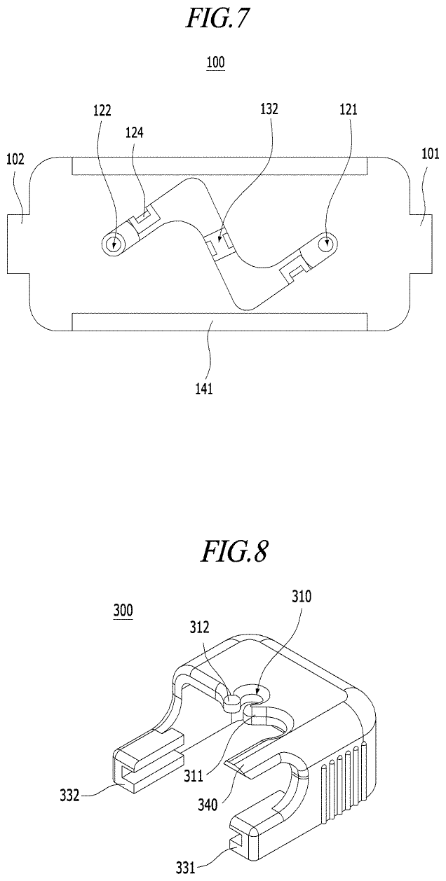

FIG. 6 is a schematic perspective view of a base part of the two-way clamping catheter stabilization device according to an exemplary embodiment of the present disclosure, and FIG. 7 is a bottom view of the base part of the two-way clamping catheter stabilization device when viewed from below according to an exemplary embodiment of the present disclosure.

Referring to FIG. 6 and FIG. 7, a punched region 132 penetrating the base part 100 in the upward and downward directions may be formed in the base part 100 in order to easily separate the connection member 230 inserted in the coupling groove 123 from the base part 100. Further, according to an exemplary embodiment of the present disclosure, there may be multiple punched regions 132 spaced apart from each other along the coupling groove 123. According to an exemplary embodiment of the present disclosure, the connection member 230 may be separated from the base part 100 by pressing the connection member 230, which has been inserted from the bottom side of the base part 100 through the coupling groove 123, in the downward direction from above the punched region 132.

Also, referring to FIG. 6 and FIG. 7, the base part 100 may include a first male rail 141 extended in the left and right directions on a front end of the base part 100 and a second male rail 142 extended in the left and right directions on a rear end of the base part 100.

Further, referring to FIG. 6, a serrated resistant member 141c that provides resistance to movement of a first female rail 331 and a second female rail 431 coupled to the first male rail 141 and moving in a longitudinal direction along the first male rail 141 and suppresses an outward separation of the first coupling part 300 and the second coupling part 400 from the base part 100 along the longitudinal direction in the locked state (in other words, suppresses a transition to the unlocked state) may be formed in a central portion of the first male rail 141 in the longitudinal direction.

Also, referring to FIG. 6, the first male rail 141 includes a first anti-retraction/opening member 141a and a second anti-retraction/opening member 141b that are formed in predetermined regions on both sides of the resistant member 141c in the longitudinal direction and protrude forwards from the base part 100, and a first anti-retraction/opening groove (not illustrated) that is dented corresponding in shape to the first anti-retraction/opening member 141a may be formed in the first female rail 331 so as to be hooked and clamped to the first anti-retraction/opening member 141a in the locked state and to maintain the locked state. Likewise, a second anti-retraction/opening groove (not illustrated) that is dented corresponding in shape to the second anti-retraction/opening member 141b may be formed in the second female rail 431 so as to be hooked and clamped to the second anti-retraction/opening member 141b in the locked state and to maintain the locked state.

Although not illustrated in the drawings, the first anti-retraction/opening member 141a, the second anti-retraction/opening member 141b and the resistant member 141c formed in the first male rail 141 and the first anti-retraction/opening groove and the second anti-retraction/opening groove formed in the first female rail 331 and the second female rail 431 may also be symmetrically provided in the second male rail 142 that is provided corresponding to the first male rail 141 on the rear end of the base part 100.

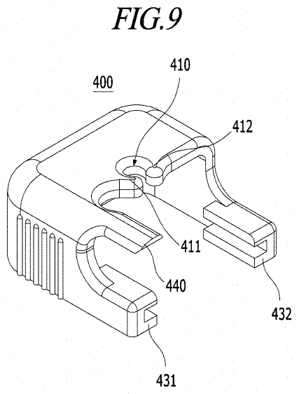

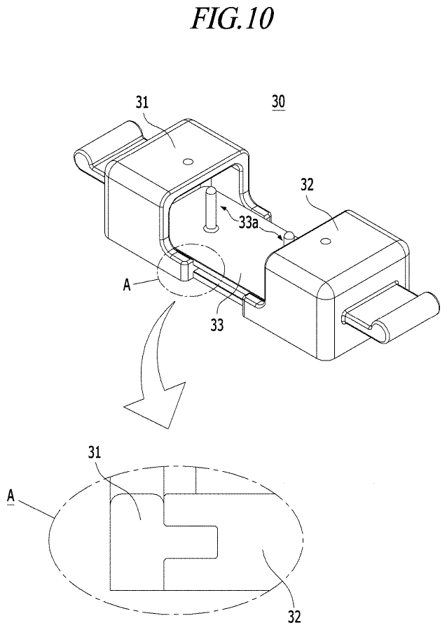

FIG. 8 is a schematic perspective view of a first coupling part of the two-way clamping catheter stabilization device according to an exemplary embodiment of the present disclosure, and FIG. 9 is a schematic perspective view of a second coupling part of the two-way clamping catheter stabilization device according to an exemplary embodiment of the present disclosure.

Referring to FIG. 8 and FIG. 9, the first coupling part 300 may include first female rails 331 and 332 corresponding to the first male rail 141 and the second male rail 142. Also, the second coupling part 400 may include second female rails 431 and 432 corresponding to the first male rail 141 and the second male rail 142.

In this regard, the first coupling part 300 may be coupled to the base part 100 so that the first female rails 331 and 332 can slide in the left and right directions along the first male rail 141 and the second male rail 142 of the base part 100. Also, the second coupling part 400 may be coupled to the base part 100 so that the second female rails 431 and 432 can slide in the left and right directions along the first male rail 141 and the second male rail 142 of the base part 100.

As described above, in the catheter stabilization device 10 of the present disclosure, each of the first coupling part 300 and the second coupling part 400 is coupled to the base part 100 by using the above-described female rails and male rails and thus can slide in the left and right directions. Therefore, the operator can fix the fixing target catheter 20 just by applying force to the first coupling part 300 and the second coupling part 400 to be closer to each other (in other words, by closing them together) and separate the mounted fixing target catheter 20 just by applying force to the first coupling part 300 and the second coupling part 400 to be farther from each other (in other words, by opening them). Accordingly, a direction of force applied by the operator to the present catheter stabilization device 10 to provide the locked state or the unlocked state may be limited to the left and right directions. Therefore, when using the catheter stabilization device 10 of the present disclosure, the operator does not need to apply force toward the body of the subject as he/she used to with a conventional catheter stabilization device. Accordingly, it is possible to suppress the application of unnecessarily great force to the subject, such as a patient or the like, and thus suppress unexpected damage to the body of the subject.

Also, referring to FIG. 8, the first coupling part 300 may include first support members 311 and 312 each protruding toward an inside in the forward and backward directions from an inlet portion of the first groove 310 so that an inlet of the first groove 310 into which the first pin 210 is to be inserted can be formed to a smaller diameter than the first pin 210. Further, referring to FIG. 9, the second coupling part 400 may include second support members 411 and 412 each protruding toward an inside in the forward and backward directions from an inlet portion of the second groove 410 so that an inlet of the second groove 410 into which the second pin 220 is to be inserted can be formed to a smaller diameter than the second pin 220.

According to an exemplary embodiment of the present disclosure, if the first pin 210 or the second pin 220 reaches the inlet portion of the first groove 310 or the second groove 410 and then further slides in a direction in which the first coupling part 300 and the second coupling part 400 become close to each other, the first support members 311 and 312 and the second support members 411 and 412 may be provided to be separated at least in part outwards in the forward and backward directions along the outer peripheral surface of the first pin 210 or the second pin 220. This means that the material or strength of the first support members 311 and 312 and the second support members 411 and 412 can be set in order for the first support members 311 and 312 and the second support members 411 and 412 to be partially bent (in other words, to be separated outwards in the forward and backward directions) so that the first pin 210 and the second pin 220 can pass through the inlet portions having a smaller diameter than the first pin 210 and the second pin 220 when moving into the first groove 310 and the second groove 410.

According to an exemplary embodiment of the present disclosure, if the first pin 210 and the second pin 220 are inserted into the first groove 310 and the second groove 410, respectively, in the locked state, the first support members 311 and 312 and the second support members 411 and 412 suppress the separation of the first pin 210 and the second pin 220 from the first groove 310 and the second groove 410, respectively, as long as extra external force for separating the first coupling part 300 or the second coupling part 400 along the longitudinal direction of the base part 100 is not applied. Further, according to an exemplary embodiment of the present disclosure, the first support members 311 and 312 and the second support members 411 and 412 may protrude in a curved shape having a predetermined curvature so that the first pin 210 and the second pin 220 can be smoothly inserted into the first groove 310 and the second groove 410, respectively.

In this regard, the locked state in which the fixing target catheter 20 mounted on the two-way clamping catheter stabilization device 10 of the present disclosure is fixed can be maintained as in “first aspect” in which the above-described first support members 311 and 312 and second support members 411 and 412 suppress the separation of the first pin 210 and the second pin 220 from the first groove 310 and the second groove 410, “second aspect” in which the anti-retraction/opening members 141a and 141b, the anti-retraction/opening grooves and the resistant member 141c suppress the separation of female rails and the male rails, and “third aspect” in which the first hook member 103 and the second hook member 104 are hooked and clamped to the first hook groove (not illustrated) of the first coupling part 300 and the second hook groove (not illustrated) of the second coupling part 400, respectively, in the locked state as long as the operator (user) does not apply a certain external force.

The above descriptions about the two-way clamping catheter stabilization device 10 can be understood from the following descriptions about a two-way clamping catheter stabilization device 30 according to another exemplary embodiment of the present disclosure in some exemplary embodiments of the present disclosure. Therefore, the descriptions about the two-way clamping catheter stabilization device 10 according to an exemplary embodiment of the present disclosure may be identically applied to the two-way clamping catheter stabilization device 30 according to another exemplary embodiment of the present disclosure, even though they are omitted hereinafter.

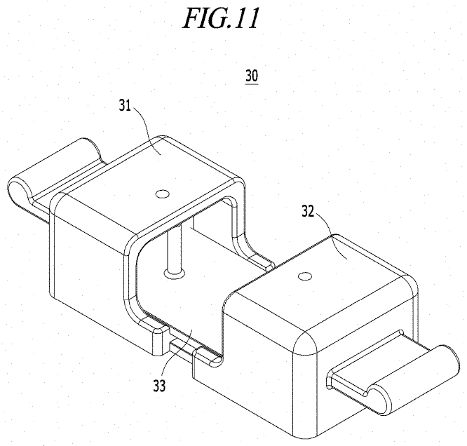

For reference, any terms referring to direction or location (top side, top surface, longitudinal direction, widthwise direction, etc.) relating to the two-way clamping catheter stabilization device 30 according to another exemplary embodiment of the present disclosure to be described below with reference to FIG. 10 to FIG. 13B are based on the placement of components shown in the drawings. For example, in FIG. 10 and FIG. 11, an upward direction may be 12 o'clock direction, a downward direction may be 6 o'clock direction, one way in a longitudinal direction may be 10 o'clock direction, the other way in the longitudinal direction may be 4 o'clock direction and a widthwise direction may be from 2 o'clock to 8 o'clock direction.

However, this direction setting may be changed depending on the placement of the device of the present disclosure. For example, the device of the present disclosure may be placed such that an upward direction in FIG. 10 and FIG. 11 faces the horizontal direction (longitudinal direction) if necessary. For another example, the device of the present disclosure may be placed such that an upward direction in FIG. 10 and FIG. 11 faces a slanted direction.

FIG. 10 is a perspective view schematically illustrating a state before a fixing target catheter is inserted into a two-way clamping catheter stabilization device according to another exemplary embodiment of the present disclosure.

Referring to FIG. 10, the two-way clamping catheter stabilization device 30 according to another exemplary embodiment of the present disclosure may include a first coupling part 31, a second coupling part 32 and a body part 33.

A fixing target catheter may be placed on a top surface of the body part 33.

Further, the body part 33 may be equipped with protrusion members 33a outside of a region where the fixing target catheter is placed in the longitudinal direction. Referring to FIG. 10, the body part 33 may be equipped with a first protrusion member in a portion leaning to one way in the longitudinal direction from the region where the fixing target catheter is placed and a second protrusion member in a portion leaning to the other way in the longitudinal direction from the region where the fixing target catheter is placed. That is, there may be two protrusion members 33a located at a distance from each other in the longitudinal direction of the body part 33. According to another exemplary embodiment of the present disclosure, the fixing target catheter may be placed in the widthwise direction of the body part 33 in the region between the first protrusion member and the second protrusion member.

Further, referring to FIG. 10, each of the protrusion members 33a may be provided in a cylindrical shape protruding upwards from the top surface of the body part 33, but may not be limited thereto.

Furthermore, the body part 33 may include a first hook member on one end in the longitudinal direction in order for the first coupling part 31 not to be separated from the body part 33, i.e., in order for the first coupling part 31 to be movable within a predetermined range along the longitudinal direction of the body part 33.

Likewise, the body part 33 may include a second hook member on the other end in the longitudinal direction in order for the second coupling part 32 not to be separated from the body part 33, i.e., in order for the second coupling part 32 to be movable within a predetermined range along the longitudinal direction of the body part 33.

The first coupling part 31 may be placed on one way in the longitudinal direction of the body part 33 and movable along the longitudinal direction of the body part 33. Herein, the first coupling part 31 is movable along the longitudinal direction of the body part 33, which means that the first coupling part 31 can move in two ways, i.e., toward the other way in the longitudinal direction so as to be closer to the center of the body part 33 in the longitudinal direction and toward one way in the longitudinal direction so as to be farther from the center of the body part 33 in the longitudinal direction.

Further, the second coupling part 32 may be placed on the other way in the longitudinal direction of the body part 33 and movable along the longitudinal direction of the body part 33. Herein, the second coupling part 32 is movable along the longitudinal direction of the body part 33, which means that the second coupling part 32 can move in two ways, i.e., toward one way in the longitudinal direction so as to be closer to the center of the body part 33 in the longitudinal direction and toward the other way in the longitudinal direction so as to be farther from the center of the body part 33 in the longitudinal direction.

That is, a distance between the first coupling part 31 and the second coupling part 32 can be changed by moving at least one of the first coupling part 31 and the second coupling part 32. According to another exemplary embodiment of the present disclosure, the first coupling part 31 and the second coupling part 32 may be moved toward the center of the body part 33 in the longitudinal direction to fix the fixing target catheter (to limit a movement of the fixing target catheter). In other words, the fixing target catheter may be fixed by decreasing the distance between the first coupling part 31 and the second coupling part 32. On the other hand, the fixing target catheter may be loosed by increasing the distance between the first coupling part 31 and the second coupling part 32.

Also, referring to an enlarged view A of a clamping structure between the body part 33 and the first coupling part 31 of the two-way clamping catheter stabilization device 30 according to another exemplary embodiment of the present disclosure as shown in FIG. 10, the body part 33 may include a female rail-form rail structure formed in one end of the body part 33 in the widthwise direction, the first coupling part 31 may include a male rail-form rail structure corresponding to the above-described female rail-form rail structure of the body part 33, the male rail-form rail structure of the first coupling part 31 may move in two ways of the longitudinal direction of the body part 33 along the female rail-form rail structure. Further, this female rail-/male rail-form coupling structure and the consequent two-way movement can be identically applied to the body part 33 and the second coupling part 32. As an example for easy understanding, the base part 100 of the two-way clamping catheter stabilization device 10 according to an exemplary embodiment of the present disclosure described above with reference to FIG. 6 includes the first male rail and the second male rail on the front end and the rear end of the base part 100 and the first coupling part 300 and the second coupling part 400 include the first female rails 331 and 332 and the second female rails 431 and 432 corresponding to the male rails, whereas the body part 33 of the two-way clamping catheter stabilization device according to another exemplary embodiment of the present disclosure includes female rail-form rail structures and the first coupling part 31 and the second coupling part 32 include male rail-form rail structures corresponding in structure to the female rail-form rail structures.

Also, an elongated hole in which one end of the body part 33 in the longitudinal direction is to be inserted may be formed in the first coupling part 31. Herein, the vertical height of the elongated hole may be set greater than the vertical height of a region of the body part 33 except the first hook member and smaller than the vertical height of the first hook member.

Likewise, an elongated hole in which the other end of the body part 33 in the longitudinal direction is to be inserted may be formed in the second coupling part 32. Herein, the vertical height of the elongated hole may be set greater than the vertical height of a region of the body part 33 except the second hook member and smaller than the vertical height of the second hook member.

Further, a first insertion hole in which the first protrusion member of the body part 33 is to be inserted may be formed in the first coupling part 31. When the first coupling part 31 moves toward the center of the body part 33 in the longitudinal direction, a top end of the first protrusion member may be inserted into the first insertion hole, and a two-way movement of the first coupling part 31 in the longitudinal direction may be limited depending on the clamping between the first insertion hole and the first protrusion member. That is, if the first protrusion member is inserted in the first insertion hole, the first coupling part 31 may be fixed to the body part 33.

Furthermore, a second insertion hole in which the second protrusion member of the body part 33 is to be inserted may be formed in the second coupling part 32. When the second coupling part 32 moves toward the center of the body part 33 in the longitudinal direction, a top end of the second protrusion member may be inserted into the second insertion hole, and a two-way movement of the second coupling part 32 in the longitudinal direction may be limited depending on the clamping between the second insertion hole and the second protrusion member. That is, if the second protrusion member is inserted in the second insertion hole, the second coupling part 32 may be fixed to the body part 33.

Referring to FIG. 10, the number of the first insertion hole and the second insertion hole may be one, but may not be limited thereto. For another example, there may be multiple first insertion holes and multiple second insertion holes in the longitudinal direction in order for the first coupling part 31 and the second coupling part 32 to be moved in stages.

FIG. 11 is a perspective view illustrating a state in which a first coupling part and a second coupling part are moved and fixed to fix the fixing target catheter inserted into the two-way clamping catheter stabilization device according to another exemplary embodiment of the present disclosure.

Referring to FIG. 11, the first protrusion member is inserted into the first insertion hole and the second protrusion member is inserted into the second insertion hole by moving the first coupling part 31 and the second coupling part 32 toward the center of the body part 33 in the longitudinal direction and thus decreasing the distance between the first coupling part 31 and the second coupling part 32. Thus, a movement of the first coupling part 31 and the second coupling part 32 relative to the body part 33 is limited and the fixing target catheter can be fixed (locked).

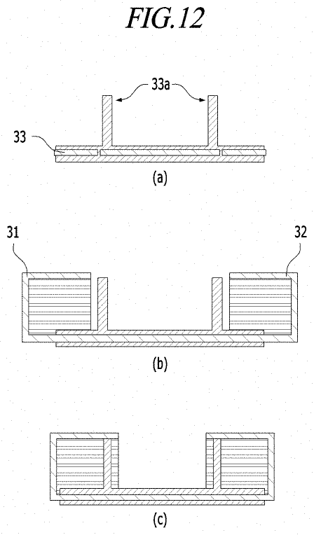

FIG. 12 is a side view of the two-way clamping catheter stabilization device when viewed from lateral side according to another exemplary embodiment of the present disclosure.

Specifically, FIG. 12A is a side view of the body part 33 of the two-way clamping catheter stabilization device 30 when viewed from lateral side, FIG. 128 is a side view of the two-way clamping catheter stabilization device 30 when viewed from lateral side before the fixing target catheter is inserted, and FIG. 12C is a side view of the two-way clamping catheter stabilization device 30 fixing the fixing target catheter when viewed from lateral side.

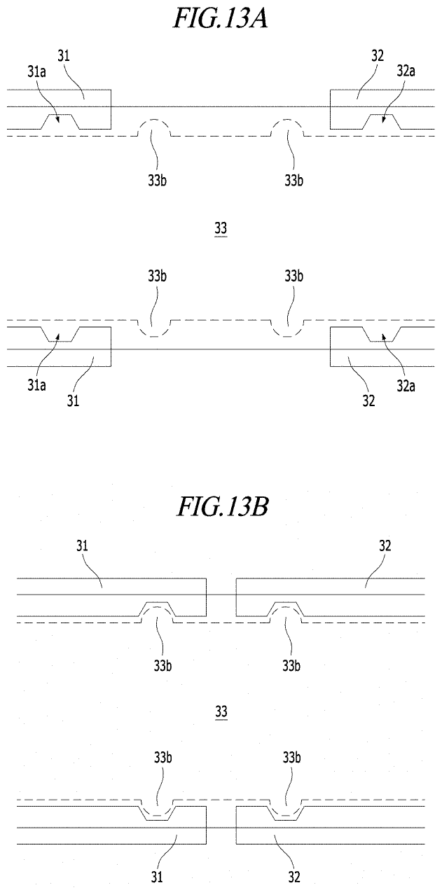

FIG. 13A is a cross-sectional view provided to explain an anti-retraction/opening structure in a state in which the fixing target catheter is not fixed by the two-way clamping catheter stabilization device according to another exemplary embodiment of the present disclosure, and FIG. 138 is a cross-sectional view provided to explain an anti-retraction/opening structure in a state in which the fixing target catheter is fixed by the two-way clamping catheter stabilization device according to another exemplary embodiment of the present disclosure.

For reference, FIG. 13A and FIG. 138 are cross-sectional views of the two-way clamping catheter stabilization device according to another exemplary embodiment of the present disclosure when viewed from above.

Referring to FIG. 13A and FIG. 138, the body part 33 may include an anti-retraction/opening member 33b protruding outwards in the widthwise direction of the body part 33 inside the female rail-form rail structure. Also, the male rail-form rail structure of the first coupling part 31 may include a first anti-retraction/opening groove 31a which corresponds in shape to the anti-retraction/opening member 33b and to which the anti-retraction/opening member 33b is to be hooked and clamped in a state in which the fixing target catheter is fixed. Likewise, the male rail-form rail structure of the second coupling part 32 may include a second anti-retraction/opening groove 32a which corresponds in shape to the anti-retraction/opening member 33b and to which the anti-retraction/opening member 33b is to be hooked and clamped in a state in which the fixing target catheter is fixed.

In other words, in a state in which the fixing target catheter 20 is fixed by the two-way clamping catheter stabilization device 30 (in the locked state), the anti-retraction/opening member 33b may be hooked and clamped to the first anti-retraction/opening groove 31a and the second anti-retraction/opening groove 32a so as to suppress the separation of the first coupling part 31 and the second coupling part 32 from the body part 33 and thus maintain the locked state as long as extra external force is not applied.

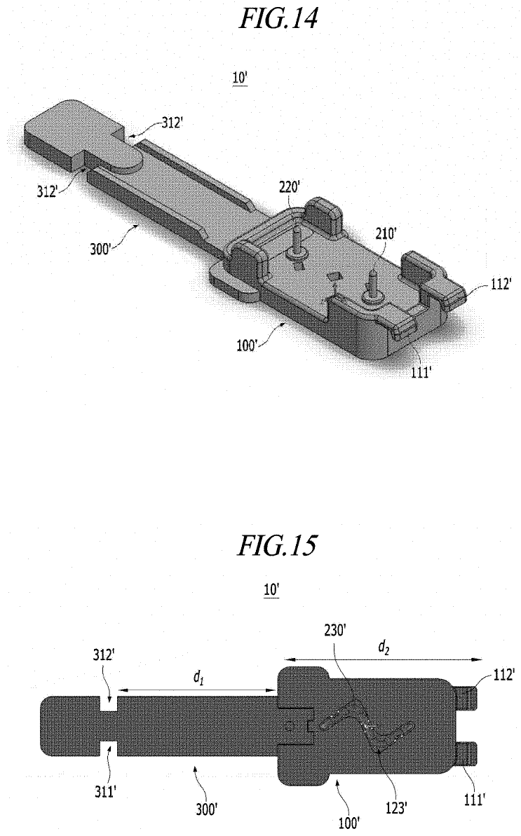

Hereinafter, a hook-type catheter stabilization device of the present disclosure will be described in detail with reference to FIG. 14 to FIG. 20.

For reference, any terms referring to direction or location (longitudinal direction, top surface, upward, upward and downward directions, widthwise direction, etc.) relating to a hook-type catheter stabilization device 10′ according to an exemplary embodiment of the present disclosure to be described below with reference to FIG. 14 to FIG. 18 are based on the placement of components shown in the drawings. For example, in FIG. 14, one way in a longitudinal direction may be 4 o'clock, the other way in the longitudinal direction may to 10 o'clock direction, a widthwise direction may be from 2 o'clock to 8 o'clock direction and upward and downward directions may be from 12 o'clock to 6 o'clock direction. Also, in FIG. 15, a longitudinal direction may be from 3 o'clock to 9 o'clock direction, a widthwise direction may be from 12 o'clock to 6 o'clock direction and upward and downward directions may be directions of normal to the drawing sheet.

However, this direction setting may be changed depending on the placement of the device of the present disclosure. For example, the device of the present disclosure may be placed such that an upward direction in FIG. 14 faces a longitudinal direction if necessary. For another example, the device of the present disclosure may be placed such that an upward direction in FIG. 14 faces a slanted direction.

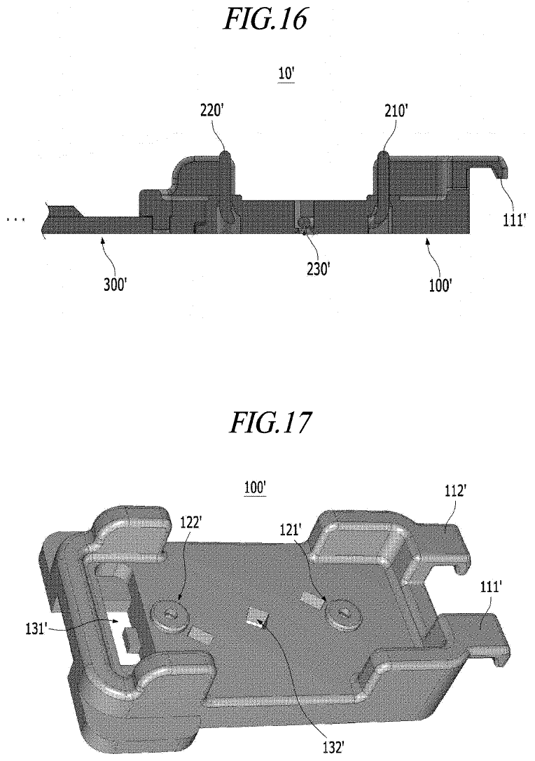

FIG. 14 is a schematic perspective view of a hook-type catheter stabilization device according to an exemplary embodiment of the present disclosure, FIG. 15 is a bottom view of the hook-type catheter stabilization device when viewed from below according to an exemplary embodiment of the present disclosure, and FIG. 16 is a side view of the hook-type catheter stabilization device when viewed from lateral side according to an exemplary embodiment of the present disclosure.

Referring to FIG. 14 to FIG. 16, the hook-type catheter stabilization device 10′ according to an exemplary embodiment of the present disclosure (hereinafter, referred to as “catheter stabilization device 10′”) may include a base part 100′, a pin part 200′ and a hook unit 300′.

The fixing target catheter 20 may be mounted on a top surface of the base part 100′. According to an exemplary embodiment of the present disclosure, the fixing target catheter 20 may be mounted on the center of the base part 100′ in the longitudinal direction so as to be extended along the widthwise direction of the base part 100′. In other words, the fixing target catheter 20 may be mounted (placed) on the catheter stabilization device 10′ so that a longitudinal direction of the fixing target catheter 20 agrees with a widthwise direction of the catheter stabilization device 10′. As an example for easy understanding, the fixing target catheter 20 may be mounted (placed) on the catheter stabilization device 10′ so that the longitudinal direction of the fixing target catheter 20 is orthogonal to a longitudinal direction of the catheter stabilization device 10′.

Further, the base part 100′ may include a pair of hook members 111′ and 112′ that protrude from one side of the base part 100′ in the longitudinal direction and are located at a distance from each other in the widthwise direction. Referring to FIG. 14 and FIG. 16, each of the pair of hook members 111′ and 112′ may protrude at least in part downwards in a ┐-shape from one side of the base part 100′ in the longitudinal direction, but may not be limited thereto.

The pin part 200′ may include a first pin 210′ and a second pin 220′ protruding upwards from the top surface of the base part 100′.

The hook unit 300′ has the other end to be coupled to the other side of the base part 100′ in the longitudinal direction and one end including a pair of hook grooves 311′ and 312′ to which the pair of hook members 111′ and 112′ are to be hooked and clamped, respectively. According to an exemplary embodiment of the present disclosure, the pair of hook grooves 311′ and 312′ may be dented from both sides of the hook unit 300′ in the widthwise direction toward the center of the hook unit 300′ in the widthwise direction so as to correspond to a distance between the pair of hook members.

Further, the hook unit 300′ may be made of a material that is bendable and elastically deformable in the longitudinal direction. Furthermore, the length of the hook unit 300′ may be set in order for the pair of hook grooves 311′ and 312′ to be located closer to the other side in the longitudinal direction than the pair of hook members 111′ and 112′ when the one end of the hook unit 300′ is moved to the one side in the longitudinal direction in a state in which the book unit 300′ is not elastically deformed in the longitudinal direction.

Specifically, referring to FIG. 15, a length d₁ in the longitudinal direction from a region where the base part 100′ is coupled to the hook unit 300′ to the pair of hook grooves 311′ and 312′ may be set smaller than a length d₂ in the longitudinal direction from the region where the base part 100′ is coupled to the hook unit 300′ to the pair of hook members 111′ and 112′ in a state in which the hook unit 300′ is not elastically deformed in the longitudinal direction based on the region where the base part 100′ is coupled to the hook unit 300′ (for example, where the other end of the hook unit 300′ is coupled to the other end of the base part 100′ in the longitudinal direction).

That is, the operator who wants to fix the fixing target catheter 20 using the catheter stabilization device 10′ of the present disclosure may hook and clamp the pair of hook members 111′ and 112′ of the hook unit 300′ to the pair of hook grooves 311′ and 312′ by moving one end of the hook unit 300′ to one side in the longitudinal direction (for example, by pulling) and applying force for elastically deforming the hook unit 300′ at least in part in the longitudinal direction. In other words, the hook unit 300′ is hooked and clamped to the base part 100′ in a state in which the hook unit 300′ is elastically deformed at least in part in the longitudinal direction, and, thus, the hook unit 300′ can be tight as compared with a state in which the hook unit 300′ is not elastically deformed in the longitudinal direction. Therefore, the fixing target catheter 20 placed (mounted) under the hook unit 300′ can be more securely supported in the downward direction. Accordingly, it is possible to effectively suppress an upward separation of the fixing target catheter 20. Further, the hook unit 300′ applying force to the fixing target catheter 20 in the downward direction is made of a material (for example, a flexible material) that is bendable and elastically deformable in the longitudinal direction. Therefore, the hook unit 300′ which can be contacted with the fixing target catheter 20 can elastically deform in an active manner depending on the size (for example, diameter or the like) of the fixing target catheter 20. Accordingly, it is possible to customize and provide fixing power (supporting power) to each of multiple fixing target catheters 20 of various sizes.

In other words, if the hook unit 300′ is elastically deformed at least in part in the longitudinal direction and one end of the hook unit 300′ is moved to one side in the longitudinal direction in order for the pair of hook members 111′ and 112′ to be hooked and clamped to the pair of hook grooves 311′ and 312′ in the catheter stabilization device 10′ of the present disclosure by setting the length of the hook unit 300′ relative to the base part 100′ as described above, a locked state in which an upward movement of the mounted fixing target catheter 20 is limited may be provided. Specifically, the locked state may be maintained by supporting a region leaning to one side of the hook unit 300′ from the pair of hook grooves 311′ and 312′ of the hook unit 300′ with the pair of hook members 111′ and 112′.

According to an exemplary embodiment of the present disclosure, the first pin 210′ and the second pin 220′ may be inserted into the holes 22 formed in the wing-type members 21 of the fixing target catheter 20. Further, according to an exemplary embodiment of the present disclosure, an upward movement of the fixing target catheter 20 may be limited by letting the wing-type members 21 to move between a bottom side of the hook unit 300′ and a top side of the base part 100′ along the first pin 210′ or the second pin 220′ in the above-described locked state (in other words, in a state in which the pair of hook members 111′ and 112′ are hooked and clamped to the pair of hook grooves 311′ and 312′).

According to an exemplary embodiment of the present disclosure, the fixing target catheter 20 may be separated from the catheter stabilization device 10′ when the wing-type members 21 of the fixing target catheter 20 are separated upwardly from the first pin 210′ and the second pin 220′ in an unlocked state in which hooking and clamping of the pair of hook grooves 311′ and 312′ and the pair of hook members 111′ and 112′ is canceled.

Furthermore, according to an exemplary embodiment of the present disclosure, a first hole 121′ in which the first pin 210′ is to be inserted and a second hole 122′ in which the second pin 220′ is to be inserted may be formed in the base part 100′. Herein, the first pin 210′ may partially protrude upwards from a bottom side of the base part 100′ through the first hole 121′. Likewise, the second pin 220′ may partially protrude upwards from a bottom side of the base part 100′ through the second hole 122′.

Further, according to an exemplary embodiment of the present disclosure, the pin part 200′ may include a connection member 230′ that connects the first pin 210′ and the second pin 220′. Furthermore, a dented coupling groove 123′ corresponding in shape to the connection member 230′ may be formed in a bottom surface of the base part 100′.

According to an exemplary embodiment of the present disclosure, the connection member 230′ of the pin part 200′ may be provided including a curved portion in at least a partial region. For example, the connection member 230′ may be provided in an S-shape between the first pin 210′ and the second pin 220′. Like the connection member 230′, the coupling groove 123′ may be provided including a curved portion in at least a partial region so as to correspond in shape to the connection member 230′.

According to an exemplary embodiment of the present disclosure, the connection member 230′ and the coupling groove 123′ corresponding to the connection member 230′ have a shape including a curved portion in at least a partial region, and, thus, even if unexpected external force is applied to a partial region of the pin part 200′ inserted into the base part 100′, the external force works along an in-plane direction of the connection member 230′ (in other words, the external force is dispersed). Therefore, it is possible to suppress leaning of the pin part 200′ to any one side or a difference in the degree of protrusion between the first pin 210′ and the second pin 220′. Eventually, it is possible to suppress the separation of the pin part 200′ from the base part 100′.

Further, according to an exemplary embodiment of the present disclosure, the base part 100′ may include at least one protrusion member 124′ configured to suppress a downward separation of the connection member 230′ inserted in the coupling groove 123′ from the base part 100′. Specifically, the protrusion member 124′ may protrude toward an inside of the coupling groove 123′ and may have a top portion that protrudes more than a bottom portion. In other words, the protrusion member 124′ may include a slanted surface in at least a partial region along the upward and downward directions. The slanted surface may be provided in order for the connection member 230′ to be easily coupled to (mounted on) the coupling groove 123′ while moving along the slanted surface from the bottom side to the top side of the base part 100′.

Furthermore, if the connection member 230′ passes through a region where the protrusion member 124′ protrudes and is inserted to be located on a top side of the protrusion member 124′, the connection member 230′ is supported upwards by the protrusion member 124′. Therefore, it is possible to suppress a downward separation of the pin part 200′ from the base part 100′.

Moreover, according to an exemplary embodiment of the present disclosure, desirably, the pin part 200′ may be made of metal at least in part in order for the first pin 210′ and the second pin 220′ not to be deformed even when the fixing target catheter 20 placed on the catheter stabilization device 10′ makes an unexpected movement.

FIG. 17 is a schematic perspective view of a base part of the hook-type catheter stabilization device according to an exemplary embodiment of the present disclosure, and FIG. 18 is a bottom view of the base part of the hook-type catheter stabilization device when viewed from below according to an exemplary embodiment of the present disclosure.

Referring to FIG. 17 and FIG. 18, a punched region 132′ penetrating the base part 100′ in the upward and downward directions may be formed in the base part 100′ in order to easily separate the connection member 230′ inserted in the coupling groove 123′ from the base part 100′. Further, according to an exemplary embodiment of the present disclosure, there may be multiple punched regions 132′ spaced apart from each other along the coupling groove 123′. According to an exemplary embodiment of the present disclosure, the connection member 230′ may be separated from the base part 100′ by pressing the connection member 230′, which has been inserted from the bottom side of the base part 100′ through the coupling groove 123′, in the downward direction from above the punched region 132′.

Also, referring to FIG. 17 and FIG. 18, according to an exemplary embodiment of the present disclosure, an opening region 131′ to which the other end of the hook unit 300′ is to be coupled may be formed on the other side of the base part 100′ in the longitudinal direction. According to an exemplary embodiment of the present disclosure, the other end of the hook unit 300′ may be inserted into the opening region 131′ and then coupled (fixed) to the base part 100′ by a support protrusion (not illustrated) protruding from a partial region on the other side of the base part 100′ in the longitudinal direction in order to support the inserted hook unit 300′. Referring to FIG. 17 and FIG. 18, the support protrusion (not illustrated) may protrude in the other way in the longitudinal direction from the other side of the base part 100′ in the longitudinal direction or protrude toward the other end of the hook unit 300′ from the bottom surface of the base part 100′ in the downward direction, but may not be limited thereto.

The above descriptions about the hook-type catheter stabilization device 10′ can be understood from the following descriptions about a hook-type catheter stabilization device 50 according to another exemplary embodiment of the present disclosure in some exemplary embodiments of the present disclosure. Therefore, the descriptions about the hook-type catheter stabilization device 10′ according to an exemplary embodiment of the present disclosure may be identically applied to the hook-type catheter stabilization device 50 according to another exemplary embodiment of the present disclosure, even though they are omitted hereinafter.

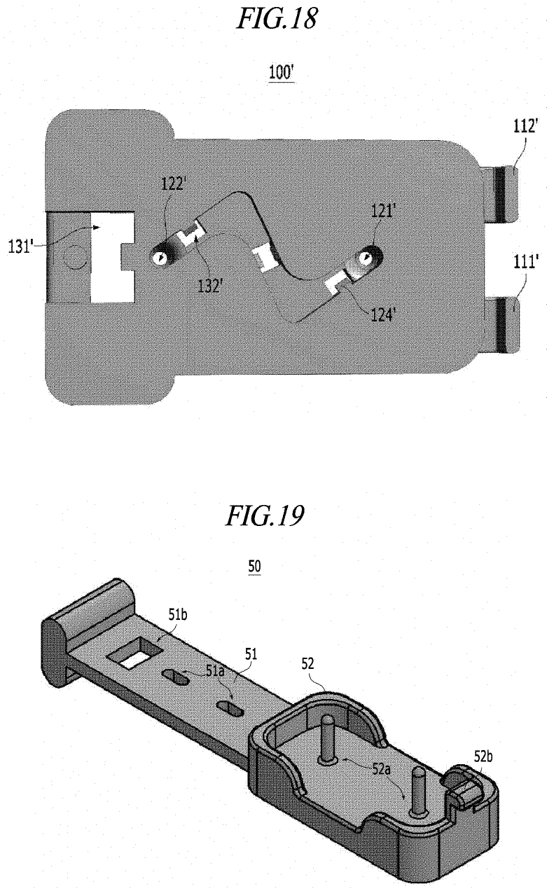

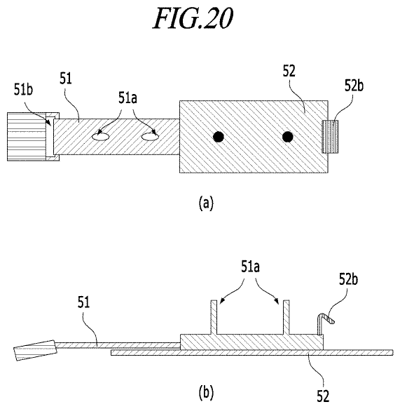

For reference, any terms referring to direction or location (top side, top surface, longitudinal direction, widthwise direction, etc.) relating to the hook-type catheter stabilization device 50 according to another exemplary embodiment of the present disclosure to be described below with reference to FIG. 19 and FIG. 20 are based on the placement of components shown in the drawings. For example, in FIG. 19, an upward direction may be 12 o'clock direction, a downward direction may be 6 o'clock direction, a longitudinal direction may be from 4 o'clock to 10 o'clock direction and a widthwise direction may be from 2 o'clock to 8 o'clock direction.

However, this direction setting may be changed depending on the placement of the device of the present disclosure. For example, the device of the present disclosure may be placed such that an upward direction in FIG. 19 faces the horizontal direction (longitudinal direction) if necessary. For another example, the device of the present disclosure may be placed such that an upward direction in FIG. 19 faces a slanted direction.

FIG. 19 is a perspective view schematically illustrating a hook-type catheter stabilization device according to another exemplary embodiment of the present disclosure.

Referring to FIG. 19, the hook-type catheter stabilization device 50 according to another exemplary embodiment of the present disclosure may include a strap part 51 and a body part 52.

A fixing target catheter may be placed on a top surface of the body part 52.

Further, the body part 52 may be equipped with protrusion members 52a outside of a region where the fixing target catheter is placed. For example, referring to FIG. 19, there may be two protrusion members 52a located at a distance from each other in the longitudinal direction of the body part 52. According to another exemplary embodiment of the present disclosure, the fixing target catheter may be placed in the widthwise direction of the body part 52 in the region between the two protrusion members 52a.

Further, referring to FIG. 19, each of the protrusion members 52a may be provided in a cylindrical shape protruding upwards from the top surface of the body part 52, but may not be limited thereto.

Furthermore, the body part 52 may include a hook member 52b. Referring to FIG. 19, the hook member 52b may protrude from an outer surface of the body part 52 toward the longitudinal direction and may be bent at least in part.

The strap part 51 may have one end connected to the body part 52 and the other end bent toward a top side of the body part 52. For example, the one end connected to the body part 52 may be one way in the longitudinal direction and the other end bent toward the top side of the body part 52 may be the other way in the longitudinal direction.

According to another exemplary embodiment of the present disclosure, the strap part 51 may be made of a flexible material having elasticity so that the strap part 51 can be bent toward the body part 52.

Further, the strap part 51 may include insertion holes 51a in which the protrusion members 52a are to be inserted. That is, if the fixing target catheter is inserted into the top surface of the body part 52 and the strap part 51 is located above the body part 52 by bending the other end of the strap part 51 toward the top side of the body part 52, the protrusion members 52a of the body part 52 can be inserted into the insertion holes 51a. In other words, it is possible to suppress the separation of the fixing target catheter from the hook-type catheter stabilization device 50 by coupling of the insertion holes 51a and the protrusion members 52a.

Further, referring to FIG. 19, the insertion holes 51a may be identical in number to the protrusion members 52a, but the present disclosure may not be limited thereto. The number of the insertion holes 51a may be changed depending on the exemplary embodiment. According to another exemplary embodiment of the present disclosure, the position of an insertion hole 51a relative to a protrusion member 52a changes as the degree of bending of the strap part 51 changes. Thus, in order for an insertion hole 51a at a different position to be coupled to the protrusion member 52a, the insertion holes 51a may be greater in number than the protrusion members 52a.

Furthermore, the strap part 51 may include a coupling member 51b to be coupled to the hook member 52b. Referring to FIG. 19, the coupling member 51b may be provided in a hole shape penetrating the strap part 51 in the upward and downward directions, but may not be limited thereto. For another example, the coupling member 51b may be bent and protruded in the same shape as the hook member 52b but bent in the opposite direction to the hook member 52b. Thus, when the strap part 51 is bent, the hook member 52b can be coupled to the bent surface of the coupling member 51b.

According to another exemplary embodiment of the present disclosure, the operator can move at least a part of the strap part 51 by pulling the other end of the strap part 51 so that the hook member 52b can be adjacent to the coupling member 51b and suppress the separation of the fixing target catheter from the hook-type catheter stabilization device 50 by fixing the hook member 52b and the coupling member 51b.

According to another exemplary embodiment of the present disclosure, in a state in which the fixing target catheter is fixed between the strap part 51 and the body part 52 by coupling the hook member 52b and the coupling member 51b, if the operator separates the coupling member 51b from the hook member 52b by manipulating the coupling member 51b to be released from the hook member 52b, the strap part 51 can be separated from the body part 52 and fixing of the fixing target catheter can be canceled.

FIG. 20 is a plan view of the hook-type catheter stabilization device when viewed from a predetermined direction according to another exemplary embodiment of the present disclosure.

Specifically, FIG. 20A is a plan view of the hook-type catheter stabilization device 50 when viewed from above and FIG. 20B is a side view of the hook-type catheter stabilization device 50 when viewed from one way in the widthwise direction.

The above description of the present disclosure is provided for the purpose of illustration, and it would be understood by those skilled in the art that various changes and modifications may be made without changing technical conception and essential features of the present disclosure. Thus, it is clear that the above-described embodiments are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description of the embodiment. It shall be understood that all modifications and embodiments conceived from the meaning and scope of the claims and their equivalents are included in the scope of the present disclosure.

We claim:

1. A two-way clamping catheter stabilization device, comprising:
a base part having a top surface on which a fixing target catheter is mounted;
a pin part including a first pin and a second pin protruding upwards from the top surface of the base part;
a first coupling part placed on a right side of the base part and coupled to the base part to be slidably moved in two ways along left and right directions of the base part; and
a second coupling part placed on a left side of the base part and coupled to the base part to be slidably moved in two ways along the left and right directions of the base part,
wherein when the first coupling part and the second coupling part slide to be closer to each other, they are placed in a locked state in which an upward movement of the mounted fixing target catheter is limited.

2. The two-way clamping catheter stabilization device of claim 1,
wherein the first pin and the second pin are inserted into holes formed in wing-type members of the fixing target catheter, and
in the locked state, the upward movement of the fixing target catheter is limited by letting the wing-type members to move between a bottom side of the first coupling part and the second coupling part and a top side of the base part.

3. The two-way clamping catheter stabilization device of claim 2,
wherein a first groove that is dented in a right direction to cover a top portion of the first pin in the locked state is formed in the first coupling part, and
a second groove that is dented in a left direction to cover a top portion of the second pin in the locked state is formed in the second coupling part.

4. The two-way clamping catheter stabilization device of claim 2,
wherein the fixing target catheter is separated when the wing-type members are separated upwardly from the first pin and the second pin in an unlocked state in which the first coupling part and the second coupling part slide to be farther from each other.

5. The two-way clamping catheter stabilization device of claim 1,
wherein a first hole in which the first pin is to be inserted and a second hole in which the second pin is to be inserted are formed in the base part, and
the first pin partially protrudes upwards from a bottom side of the base part through the first hole and the second pin partially protrudes upwards from a bottom side of the base part through the second hole.

6. The two-way clamping catheter stabilization device of claim 5,
wherein the pin part includes a connection member that connects the first pin and the second pin, and
a dented coupling groove corresponding in shape to the connection member is formed in a bottom surface of the base part.

7. The two-way clamping catheter stabilization device of claim 6,
wherein the connection member is provided including a curved portion in at least a partial region.

8. The two-way clamping catheter stabilization device of claim 6,
wherein the base part includes a protrusion member configured to suppress a downward separation of the connection member inserted in the coupling groove from the base part.

9. The two-way clamping catheter stabilization device of claim 8,
wherein the protrusion member protrudes toward an inside of the coupling groove and has a top portion that protrudes more than a bottom portion.

10. The two-way clamping catheter stabilization device of claim 1,
wherein the base part includes a first support member protruding in a right direction on a lower right side of the base part and a second support member protruding in a left direction on a lower left side of the base part, and
the first support member includes a first hook member that protrudes upwards, and a first hook groove that is to be hooked and clamped to the first hook member in the locked state is formed in the first coupling part, and
the second support member includes a second hook member that protrudes upwards, and a second hook groove that is to be hooked and clamped to the second hook member in the locked state is formed in the second coupling part.

11. The two-way clamping catheter stabilization device of claim 10,
wherein the base part includes a first male rail extended in the left and right directions on a front end of the base part and a second male rail extended in the left and right directions on a rear end of the base part, and
the first coupling part includes a first female rail corresponding to the first male rail and the second male rail, and
the second coupling part includes a second female rail corresponding to the first male rail and the second male rail, and
the first coupling part is coupled to the base part in order for the first female rail to be slidably moved in the left and right directions along the first male rail and the second male rail, and
the second coupling part is coupled to the base part in order for the second female rail to be slidably moved in the left and right directions along the first male rail and the second male rail.

12. The two-way clamping catheter stabilization device of claim 11,
wherein the first coupling part includes a first limit block protruding toward the left direction on a top portion of the first coupling part, and
the second coupling part includes a second limit block protruding toward the right direction on a top portion of the second coupling part, and
the first limit block and the second limit block are provided to suppress an upward separation of the fixing target catheter in the locked state.

13. The two-way clamping catheter stabilization device of claim 12,
wherein the first limit block and the second limit block protrude from a region leaning from forwards or backwards center in forward and backward directions of the top portion of the first coupling part and the top portion of the second coupling part, respectively.

14. A hook-type catheter stabilization device, comprising:
a base part having a pair of hook members that protrude from one side in a longitudinal direction and are located at a distance from each other in a widthwise direction;
a pin part including a first pin and a second pin protruding upwards from a top surface of the base part; and
a hook unit having the other end to be coupled to the other side of the base part in the longitudinal direction and one end including a pair of hook grooves to which the pair of hook members are to be hooked and clamped, respectively,
wherein the hook unit is made of a material that is bendable and elastically deformable in the longitudinal direction, and
a length of the hook unit is set in order for the pair of hook grooves to be located closer to the other side in the longitudinal direction than the pair of hook members when the one end is moved to the one side in the longitudinal direction in a state in which the hook unit is not elastically deformed in the longitudinal direction.

15. The hook-type catheter stabilization device of claim 14,
wherein when the one end of the hook unit is moved to the one side in the longitudinal direction in order for the hook unit to be elastically deformed at least in part in the longitudinal direction and for the pair of hook grooves to be hooked and clamped to the pair of hook members, a fixing target catheter mounted on the top surface of the base part is placed in a locked state in which an upward movement of the fixing target catheter is limited.

16. The hook-type catheter stabilization device of claim 15,
wherein the pair of hook grooves are dented toward the center of the hook unit in the widthwise direction in order to correspond to the distance between the pair of hook members.

17. The hook-type catheter stabilization device of claim 16,
wherein in the locked state, a region leaning toward the one side of the hook unit from the pair of hook grooves of the hook unit is supported by the pair of hook members to maintain the locked state.

18. The hook-type catheter stabilization device of claim 15,
wherein the first pin and the second pin are inserted into holes formed in wing-type members of the fixing target catheter, and
in the locked state, the upward movement of the fixing target catheter is limited by letting the wing-type members to move between a bottom side of the hook unit and a top side of the base part.

19. The hook-type catheter stabilization device of claim 18,
wherein the fixing target catheter is separated when the wing-type members are separated upwardly from the first pin and the second pin in an unlocked state in which hooking and clamping of the pair of hook grooves and the pair of hook members is canceled.

20. The hook-type catheter stabilization device of claim 14,
wherein a first hole in which the first pin is to be inserted and a second hole in which the second pin is to be inserted are formed in the base part, and
the first pin partially protrudes upwards from a bottom side of the base part through the first hole and the second pin partially protrudes upwards from a bottom side of the base part through the second hole.

21. The hook-type catheter stabilization device of claim 20,
wherein the pin part includes a connection member that connects the first pin and the second pin, and
a dented coupling groove corresponding in shape to the connection member is formed in a bottom surface of the base part.

22. The hook-type catheter stabilization device of claim 21,
wherein the connection member is provided including a curved portion in at least a partial region.

23. The hook-type catheter stabilization device of claim 21,
wherein the base part includes a protrusion member configured to suppress a downward separation of the connection member inserted in the coupling groove from the base part.

24. The hook-type catheter stabilization device of claim 23,
wherein the protrusion member protrudes toward an inside of the coupling groove and has a top portion that protrudes more than a bottom portion.
